(19) United States
(12) Patent Application Publication
(10) Pub. No.: US 2017/0014300 A1
(43) Pub. Date: Jan. 19, 2017

(54) Moldable heater with Miniature Resonant Frequency Vibration Generator for Ophthalmic Eyelid Therapy

(71) Applicants: James Lyle Dippo, Arvada, CO (US); Barry Johnathan Linder, Danville, CA (US); Ronald D. Linder, Gilbert, AZ (US); Rubin DeGuang Linder, Danville, CA (US); John A. Scholl, San Ramon, CA (US)

(72) Inventors: James Lyle Dippo, Arvada, CO (US); Barry Johnathan Linder, Danville, CA (US); Ronald D. Linder, Gilbert, AZ (US); Rubin DeGuang Linder, Danville, CA (US); John A. Scholl, San Ramon, CA (US)

(73) Assignee: EyeDetec Medical, Inc., Danville, CA (US)

(21) Appl. No.: 15/187,457

(22) Filed: Jun. 20, 2016

Related U.S. Application Data
(60) Provisional application No. 62/230,843, filed on Jun. 18, 2015.

(51) Int. Cl. A61H 23/02; A61F 7/03

(52) U.S. Cl. CPC A61H 23/02; A61F 7/034; A61F 2007/0004

(57) ABSTRACT

A combined eyelid warming device, resonance frequency generator, coupling device, and tissue response sensor are disclosed in the field of use in dry eye treatment. These combined devices are used to ensure that the entire eyelid surface and periorbital structures receive defined therapeutic warmth and appropriately tuned harmonic resonance frequency for a required period of time to stimulate flow from the Meibomian glands. The combined elements of the invention include an easily moldable heating disc, targeted formulation of the heating material, miniaturized resonant frequency vibrating generator combined with a properly designed reusable mask, to ensure precise location of the heat. Vibration may be delivered continuously or intermittently, and may be delivered according to one or more patterns. A chemical sensor is included to characterize flow as vibration is varied. The external energy source allows for circuitry in the external power module to act as a sweep range device, targeting the specific optimally tuned point or sweep frequency the individual patients body reacts to during initial use. The initial testing device supplied to the Doctor for treatment would include sensing elements to determine the optimal harmonic resonance frequency. Once the frequency is defined, the patient would receive a kit with custom harmonic resonant frequency vibrating generators set to their optimum parameters. This configuration would be amenable to both single use and reusable heater approaches.

### BENEFIT CLAIM

Provisional Patent Application No. 62/230/843 with a filing date of Jun. 18, 2015 also found in the Application Data Sheet. U.S. Pat. No. 8,721,572 B1, issued May 13, 2014, Linder et al).

### STATEMENT AS TO FEDERALLY SPONSORED RESEARCH

No government contract or public funds were used to develop this device.

### BACKGROUND OF THE INVENTION

Eye patch devices are known in the art. For example, U.S. Pat. No. 4,682,371 discloses a protective eye patch. The '371 patch has several tabs for securing the patch to a patient's eye. U.S. Pat. No. 3,068,863 discloses a patch designed to keep the eye closed. U.S. Pat. No. 3,092,103 discloses a patch with a cushion material at the edge that allows the patient's eye to move underneath the eye patch. U.S. Pat. No. 3,908,645 for an ophthalmic therapeutic pressure bandage with a conformable, permeable carrier tape.

U.S. Pat. No. 6,409,746 discloses an eye pillow that releases steam from its surface applied to the eyes and the area around the eyes. The temperature is 50° C. or lower and has a total weight of 50 g or more.

Several conditions exist for which medical and cosmetic therapy is appropriate. For example, blepharitis, meibomitis, chalazia, and/or styes are common disorders of the eyelids that cause chronic inflammation in the peri-orbita, and are often associated with ocular tear film abnormalities resulting in dry eye symptoms. Symptoms of dry eye disease and blepharitis include burning, itching, light sensitivity, blurred vision, and foreign body sensation. Signs include eyelash crusting, ocular discharge, eyelid scaling and swelling, corneal staining, and redness. For example, staphylcoccal blepharatis can have scaling and crusting along the eye lashes. There is no cure for blepharitis, and long term treatment is required to keep it under control. The following terms can all be used interchangeably throughout this invention; dry eyes, dry eye disease, dry eye syndrome, evaporative dry eye, lipid deficiency dry eyes, blepharitis, meibomian gland disease, and Meibomian gland dysfunction.

The predominant cause of dry eye is an insufficient lipid layer of the surface of the tear film. In a healthy eye, this oily layer inhibits the evaporation of the water based sub layers of the tear film maintaining a stable tear film. These lipids are produced in the Meibomian glands located in the eye lids. For those suffering dry eye disease, the likely root cause is Meibomian glands that have become clogged and a reduced quantity of lipids flowing out to the tear film.

Currently available treatments for dry eye disease and related conditions include warm compresses, such as a warm washcloth, that heat the debris and crust on the lid for 5-10 minutes. After the lid has been warmed, a lid scrub is performed by using a suitable soap, such as Neutrogena or Johnson's Baby Shampoo. Commercially available cleansing pads are available to assist in performing the lid scrub, for example OCuSOFT® Lid Scrubs or Novartis Ophthalmics Eye Scrub®. Following the eye scrub, antibiotics, such as polysporin, tobramycin, or erythromycin can be applied, to alleviate patient discomfort. Another condition for which therapy is appropriate is meibomitis, also known as the meibomian gland dysfunction. Meibomitis is a dysfunction of the meibomian gland that provides an oily layer as a critical component of the eye's natural tear film.

As reported by Olson et al, of the Schepens Eye Institute in Boston, warm moist compress therapy applied to the skin of the closed eyelids increases tear-film lipid layer thickness for subjects with meibodian gland dysfunction by more than 80% 5 minutes after initiating treatment and an additional 20% after 15 minutes of treatment. (See, OLSON, et al., Increase in Tear Film Lipid Layer Thickness Following Treatment with Warm Compresses in Patients with Meibomian Gland Dysfunction, Eye Contact Lens 29(2):96-99 (2003) available from Pub Med PMID 12695712). The transition temperature from a solid to a liquid for meibomian lipids is actually a range from 28° C. to 32° C. because of an individual's mixture of lipids. The temperature of the eyelids will therefore affect the liquidity of meibomian lipids and hence their viscosity. The non-Newtonian lipid mixture is known to undergo shear thinning when exposed to shear forces.

Further it is found that oscillations enhance the flow rate of a shear-thinning fluids. (See The International Workshop on Meibomian gland. Dysfunction: Report of the Subcommittee on Anatomy, Physiology, and Pathophysiology of the Meibomian Gland, Erich Knop, et al, Investigative Ophthalmology & Visual Science, Special Issue 2011, Vol. 52, No. 4, 2011 and The Effect of Vibration on Flow Rate of Non-Newtonian Fluid, MA Zaeem, et al, SIAM.)

Conventional ocular heating devices, such as warm compresses, typically require an external power source. These sources include electricity, a stove top boiling preparation, or a microwave appliance, and are difficult to provide a controlled temperature to the eyelids, are labor intensive, cumbersome, and inconvenient, and therefore historically result in poor patient compliance and persistence with the recommended therapy. Some success is realized with in-office, Doctor assisted visits. What is needed is a convenient, accurate, and effective, easily used hand moldable heating source that patients or their doctors apply via a coupling mechanism to patient's eyelids, and which delivers a therapeutic temperature to the entire eyelid surface independent of the individual's orbital anatomy, for a sufficient length of time to be effective.

In conjunction with the heating benefit a miniature resonant frequency vibrating generator, tuned to the patient's particular eyelid anatomy for optimal response, will stimulate Meibomian lipid flow (Provisional Patent Application File No: 9278.10900, Linder et al, and U.S. Pat. No. 8,721,572 B1, issued May 13, 2014, Linder et al). A fundamental innovative advancement utilizing both components in one beneficial delivery system is needed for the treatment of dry eye and Meibomian gland disease.

There is also a need for a device or component of the system that incorporates a moldable gel material to serve as a coupling element, heated and able to deliver heat and resonance frequency vibration to the target tissue of the eyelid, as well as to detect a positive eyelid resonance response from a broad range of generated harmonic frequencies, thus allowing a personalized or custom approach to each individual user.

### SUMMARY

The present disclosure describes a reusable system and one-time use or reusable components for a heating device with a reusable miniature harmonic frequency generator inside a reusable eye mask with a coupling device. The design ensures proper delivery of targeted heat therapy and appropriately tuned harmonic resonance frequency to the location of the eyelids and over their entire eyelid surface. This will unblock clogged Meibomian glands and ducts and encourage flow of meibomia lipid to the surface of the eye and into the tear film.

The flexible and moldable three dimensional shape of the warmer allows desired, uniform heat transfer to the area stated while the composition of the heater controls the temperature and desired length of time heat is converted and delivered to the ocular surface. The reusable miniature resonant frequency vibration generator is set to a specific patient frequency to encourage the production and flow of natural tear lipid component from the Meibomian glands in the eyelids. In addition to warming the eyelid surface and Meibomian glands directly, the heat to the surrounding periorbital area increases vascular perfusion and thereby naturally increases tissue temperature. Thusly heated, the viscosity of the lipid in the Meibomian glands is decreased.

The addition of harmonic resonance frequency generation and applied shear forces tuned to the patient's harmonic resonance of their eyelids and Meibomian gland encourages flow of the lipids expressed in these glands, delivering this critical tear film lipid layer onto the ocular surface.

An alternate and preferred embodiment of the above is to place an externally powered heater in the mask along with the externally powered harmonic frequency generator. The external energy source allows for circuitry in an external power module to act as a sweep range device, targeting the specific optimal point or sweep frequency to be tuned to the individual patient's body during initial testing by the Doctor. The initial testing device would be supplied to the Doctor and would be a plug in device with sensing elements to identify the optimally tuned harmonic resonance frequency. Once the frequency is defined and treatment provided in the Doctors' office, the patient would optionally receive a reusable mask with custom harmonic resonant frequency vibration generators set to match Doctor's test optimized treatment parameters. This configuration would be amenable to both single use and reusable heating disc approaches.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a front view of a heating disc device; FIG. 1B is a side or section view of the heating disc.

FIG. 2A is a front view of a packaged heating disc; FIG. 2B is a section view of a packaged heating disc.

FIG. 3A is a front view of the heating disc with attached resonant frequency generator; FIG. 3B is a side view of the heating disc with attached resonant frequency generator and USB connector.

FIG. 4A and FIG. 4B are a side section view and front section view heater disk showing embedded metallic nano-particles.

FIG. 5A and FIG. SB are a side section view and front section view heater disk showing embedded metallic and ceramic nano-particles.

FIG. 6A is a reusable mask incorporating both a miniature resonant frequency vibrating generator and permanent imbedded heater element; FIG. 6B shows an example vibration modulation controller; FIG. 6C shows an example mechanical resonator; FIG. 6D shows an example control board.

FIG. 7 is a table of porous plastic and moldable materials.

FIGS. 8A-8F are graphs of the force velocity and differential pressure for various porous polyolefin compositions.

FIG. 9A is a section view of a microfluidic sensor, FIG. 9B is a front view of a microfluidic sensor.

FIG. 10A is a schematic for a dual channel solution sensing electrode; FIG. 10B is a schematic for a single channel solution sensing electrode.

FIG. 11 is a representative far infrared front end spot heater.

### DETAILED DESCRIPTION

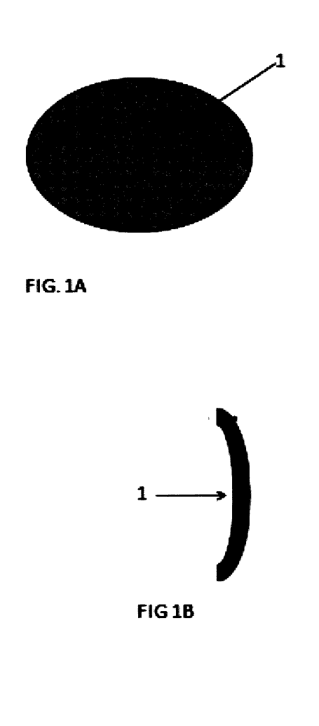

Referring now to the disclosed devices in more detail, in FIG. 1A and FIG. 1B there is shown a heating disc 1 manufactured to fit comfortably in a reusable mask conforming to the natural shape of the closed eyelid surface, and surrounding periorbital area. A suitable shape is an oval configuration better describing the shape of the ocular surface, but can also be manufactured in rectangle or square shapes that are bigger than this minimum oval requirement.

In further detail, still referring to the devices disclosed in FIG. 1A and FIG. 1B, the heating disc 1 shows a side view of an initial curvature provided to conform the device to the ocular surface by the patient when in use. The device is manufacturable such that it has a first configuration, such as an initial curvature as shown, and is conformable by the patient, physician or technician into a second configuration when in use. The material is pliable and can be easily shaped to maximize the contact area between mask and the ocular surface, to accurately match an individual user's particular orbital anatomy. For example, there will be a difference in shape for a user with a deeply set eye and deep orbit, versus a user with an anteriorly placed globe. The heating disc can be shaped by hand to fit a particular orbital anatomy. This approach will accurately match an individual user's particular orbital anatomy with the heating disc's high shape retention characteristics. Alternatively light compression from the reusable eye mask will conform the heating disc to the surface of the eye.

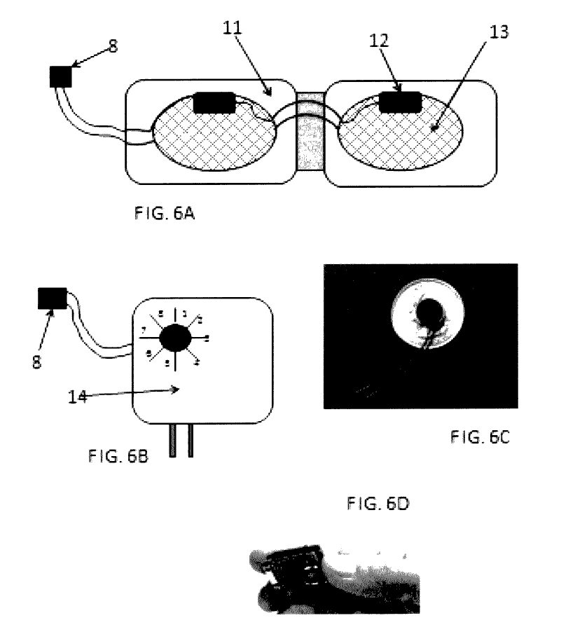

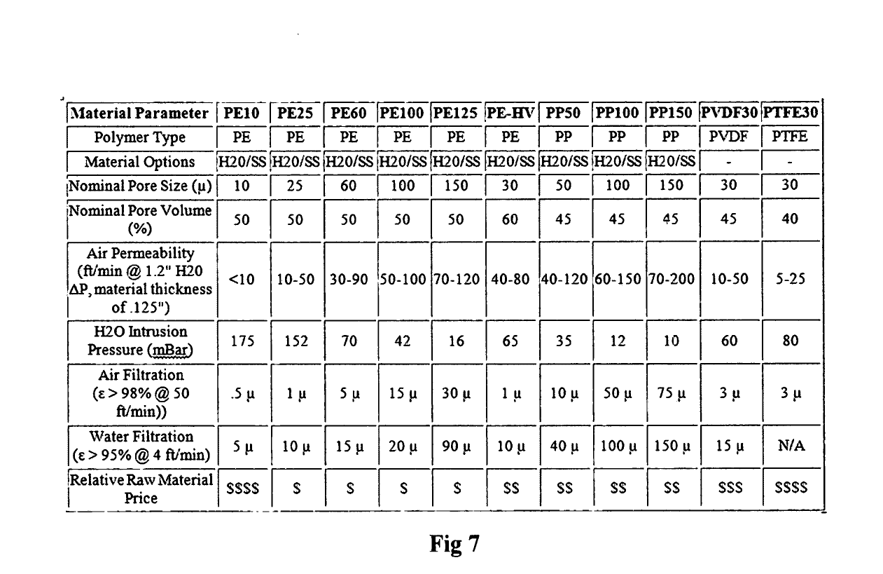

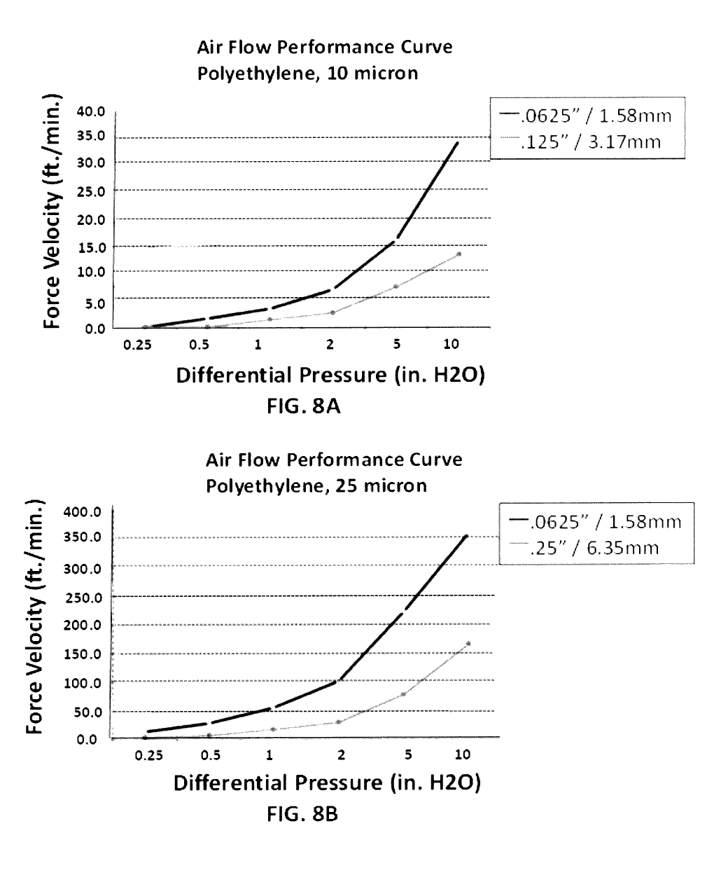

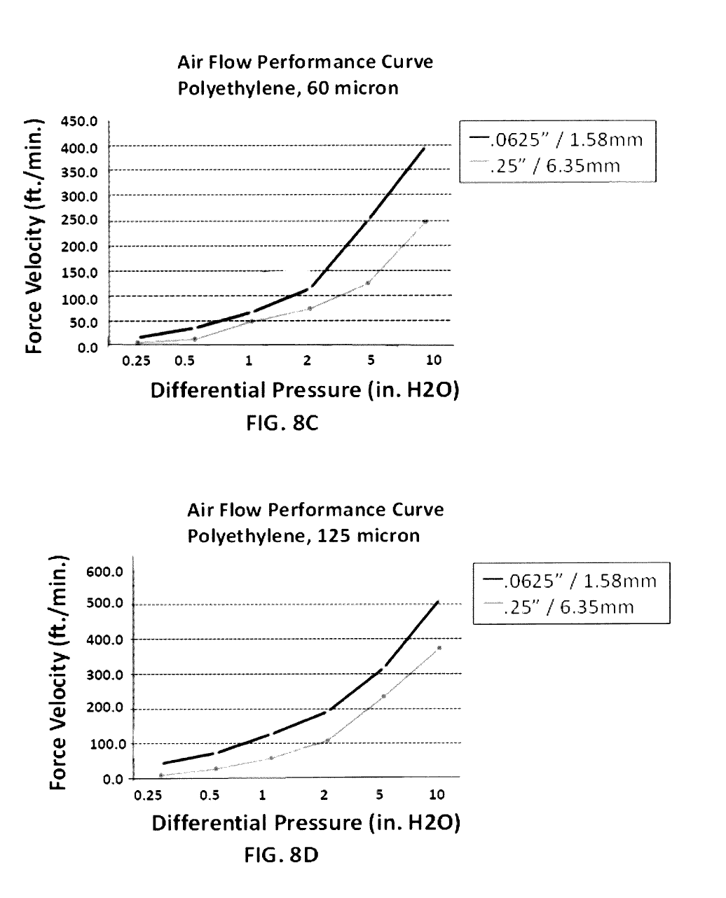

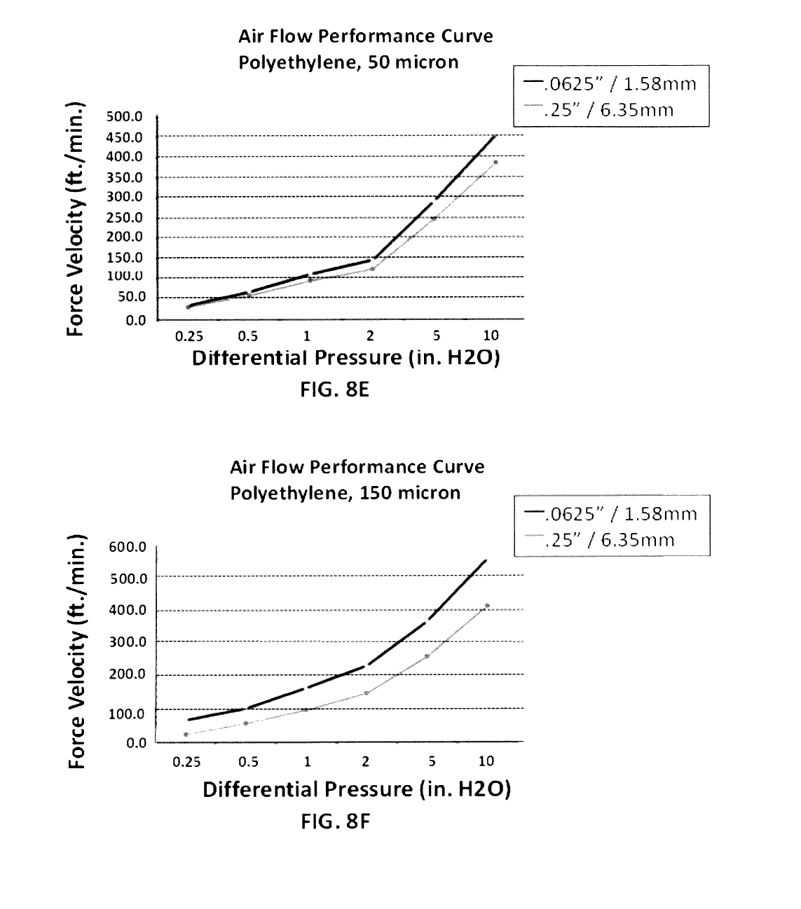

The desired heating disc integrity and heating duration are achieved by controlling disc thickness, formulation of the heating material, and porosity that allows controlled air flow to the heating material. Ideal time for application of heat is in a range from 5 to 30 minutes, preferably from 10 to 20 minutes and temperature at the surface of the eye lid should be between 40 and 46C. Example polyethylene based materials with a usable 25-60 μm pore size (PE25 through PE60) are shown in FIG. 6.

A nominal pore volume of 50% will allow the heating disc to be reshaped or molded by the patient. The porous and moldable polyethylene based material (PE) can have nominal pore sizes of 7-150 micrometers and are manufactured up to 300 micrometers in pore size. Another Polyolefin material, Polypropylene, (PP-100 and PP150), shown in FIG. 6, is a heating disc material with 100-150 μm pore size with a smaller 45% pore volume can be infused with larger heater material particles for a longer disc heating time of 20-25 minutes.

The material porosity allows a heating material to reside in the pathways with access to air at between 10-90 ft/min @ 1.2″ H₂O ΔP, where the material is between 0.125″ (3.175 mm) and 0.250″ (6.35 mm) thick with enough porosity space to adhere sufficient heating material to the support surface and internal sites. The construction details of the heating disc shown in FIG. 1A and FIG. 1B are that the heating disc 1 material be made of iron converting to ferrous oxide, with salts and inert materials resulting in the following reaction (4Fe(s)+3O₂(g) >>2Fe₂O₃(s). The salts and inert materials act as reaction accelerants or retardants as needed to slow or speed the reaction and as dispersants to help create a uniform mixture within the polymer matrix. Once the reaction of 4Fe(s) +3O₂(g) >>2Fe₂O₃(s) is driven to completion the individual reaction cannot easily be reversed, so each disk becomes a single use disposable product.

The heating disc 1 may be made of a broad combination of the ingredients resulting in a sufficiently rigid and strong molded material that can hold its shape, yet is easily hand moldable to the ocular surface for optimal therapeutic effect. The material porosity allows the heating material to reside in the pathways with access to air at between 10-90 ft/min @ 1.2″ H₂O ΔP, where the material is between 0.125″ (3.175 mm) and 0.250″ (6.35 mm) thick with enough porosity space to adhere sufficient heating material to the support surface and internal sites. Further, the various ingredients of the disc can be substituted for different materials by shape and size to control the heating rate, total thermal energy converted and delivered, and longevity of the heat conversion.

Referring now to FIGS. 1A & 1B, there is shown the heating disc 1 with the curvature. Although the disc appears to be solid in nature, there is porosity available to allow access of air to the surface and orifices of the heating material. The chosen porosity along with the ingredient choices and density of the disc when manufactured, and available surface area of the heating material, dictates heat conversion rates.

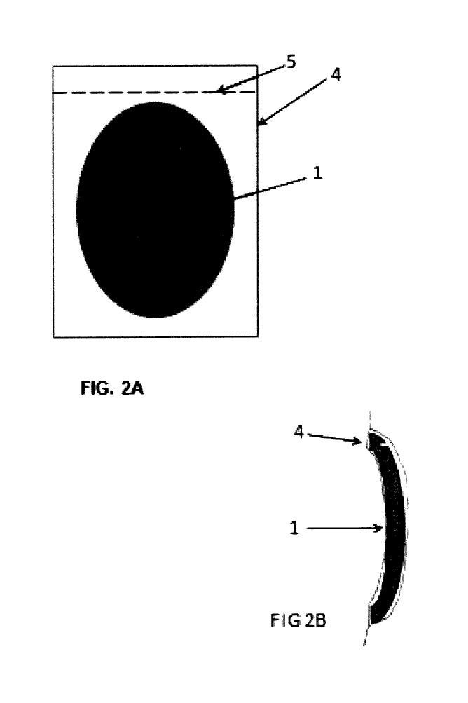

In more detail, still referring to the heating disc, the disc shown in FIG. 2A & 2B includes an overwrap material or barrier layer 4 for shipping and product storage until used by the patient. The barrier layer provides an extended shelf life of 3 years and is needed to keep H₂O and air away from the heating disc to prevent premature reaction to the heating material. Water Vapor Transmission Rate (WVTR) measures the transmission of water vapor through a material and is determined using a modulated Infrared Sensor ASTM F1249 Test Procedure. WVTR is measured in either grams/100 in²/24 hours or grams/m during 24 hours (according to the standard ASTM-E398). Oxygen Transmission Rate (OTR) is the measurement of the amount of oxygen gas that passes through a material over a given period of time and is determined using a Coulometric Sensor ASTM D3985, ISO 15105 Test Procedure. OTR is measured in either cm³/m²/24 hours or cm³/100 in²/24 hours. The barrier layer preserves the desired properties of the disc for at least three years. Lower OTR and WVTR rates are preferred. An example of materials with good storage properties are WVTR rates of 0.3 g/100 in²/24 hours @ 37.8° F.=90% RH, for Polypropylene and 0.35 g/100 in²/24 hours @ 37.8° F.=90% RH, for HDPE. While very good for WVTR, these have less attractive OTR rates at 150 cm³/100 in²/24 hours @ 25° C., for Polypropylene and 110 cm³/100 in²/24 hours @ 25° C., for HDPE. Typical long term barrier layers also include a layer of aluminum foil. This is a good example of why we use multi-layer commercial films for the product overwraps which include preferred transmission rates for both WVTR and OTR characteristics. When combined, these characteristics achieve longer shelf lives for three year protection of a product sensitive to water and oxygen.

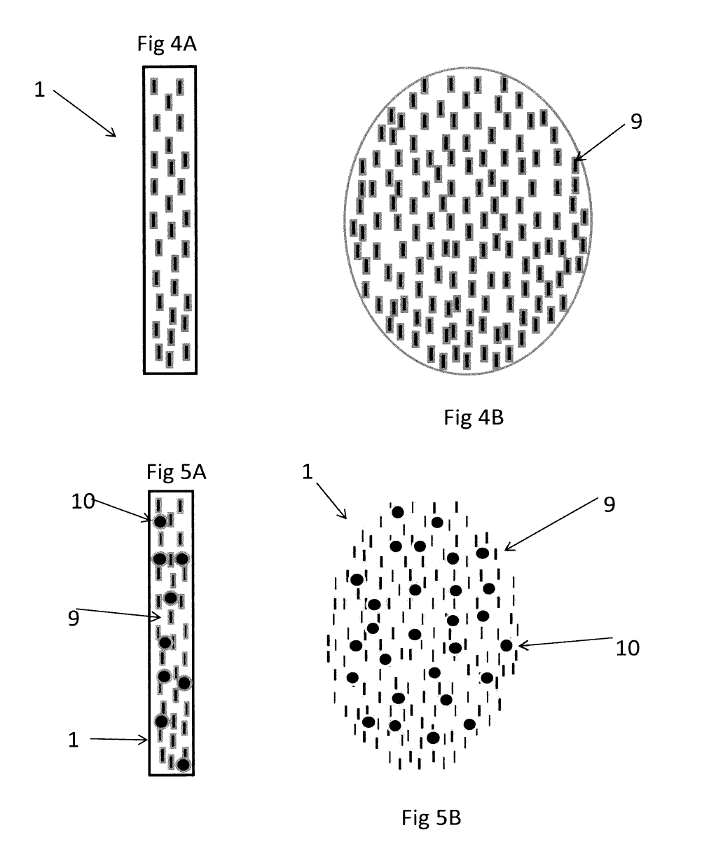

In further detail, still referring to the heating disc of FIGS. 4A-B and FIGS. 5A-B the construction of the disc, meaning its shape, materials of construction, ingredient ratios of construction material, surface area, density and porosity make this disc a unique moldable heater product. Furthermore, the heating disk polymer support material, during manufacture, can be infused with nano-particles of silica or metals to store and then release the heat generated by the FeOₓ heating material. This combination of components in the polymer matrix can be titrated and calibrated to transfer heat rate across the supporting materials and mask at a desired rate. Nano-particles of metals 9 are good heat conductors where the polymer is not. Nano-particles of silica or ceramic 10 act as a heat sink to store and slowly release the heat energy. Adding nano-particles to the polymer, particularly the metals, also enhances desirable vibration transfer characteristics. The nano-particles participate in structural integrity, also enabling a degree of structural pliability that allows for customized molding of the product to the individual patient's ocular cavity, as well as greater longevity of use for the product. This also translates into better product survival during transportation and during customer use. Polymer choices with good flexibility, shape memory, permeability and good heat transfer for desirable product characteristics can be determined from the table in FIG. 6.

A preferred embodiment for a heating mask with harmonic resonant frequency vibration is designed for use or reuse in a treating physician's office. Referring to FIG. 6A the reusable mask 11 contains a built in heater element 13 or individual elements for each eye. The system further includes resonant frequency vibration generators (RFVG) 12 to transfer shear forces or vibrational energy to the surface of the eyes. Those RFVGs could be a single unit or again one for each eye. The reusable mask 11 is designed to hold these components and fit comfortably over the patient's head and apply gentle pressure to the closed eye lids. To transfer the energy (heat and vibration) directly to the eye lid surface a sanitary and disposable single use coupling device contacts the patient's skin. Energy and control is provided to the mask via a micro USB 8 or other suitable connector from a mobile control module.

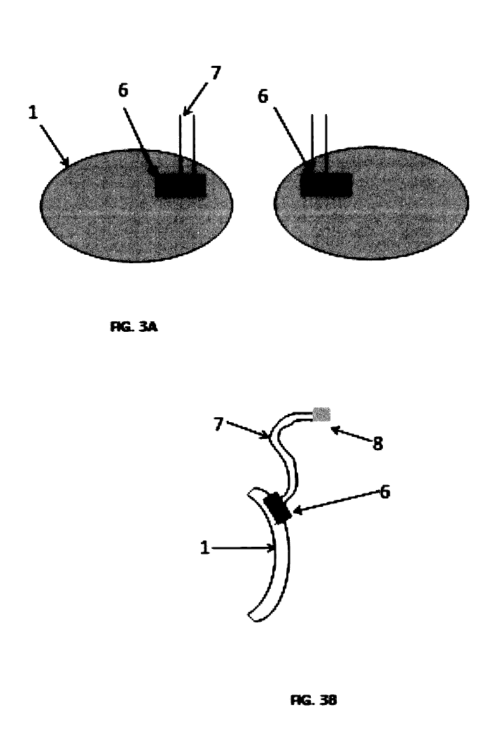

The detail of the re-usable mask in FIG. 3A shows a built in resistance heater element 13 or metal wire woven into the mask designed with a heating element length to convert enough electrical input to the appropriate eye lid at optimum temperatures as detailed above. The resistance heater wire or metal wire can be woven into the fabric or structure of the reusable mask and has dual purposes: one, convert to heat when electrically energized and, two, serve as a good vibration conductor.

Power supply to the resistance heater can be accomplished for example through the USB connection 8 to a micro drive or mobile control module. Alternate power sources include disposable and rechargeable batteries. These batteries could be placed into the reusable mask if desired to eliminate cords extending from the reusable mask. A micro drive control board controlling the heater and resonator functions could be powered from a single supply voltage of 8-48VDC, offering up to 100 W of peak power without any additional heat-sink. FIG. 6D is an example control board sold by Ingenia Motion Control. These boards combine the controller, drive and stand-alone capability into a single unit with an incredibly small footprint. The control board preferably resides in a mobile system controller that is easy to carry around the physician's office. The mobile controller might combine the controller, drive and stand-alone capability into a single unit with a small footprint. This is but one example of “off the shelf” components available to combine with the reusable mask and complete the frequency set-up and control of the reusable mask.

The miniature resonant frequency vibration generator 12 induces a vibration through the coupling device to the surface of the eye lid. The control of vibration may include amplitude, a width, frequency and where one or more of these parameters may be varied over the treatment period. The resonant vibration may have a frequency between about 2 Hz to about 270 Hz, between about 15 Hz to about 40 Hz, or between 30 Hz to about 60 Hz. The resonant vibration may include a current having a pulse width or duty cycle between about 20% to about 80%. Vibration having the above-mentioned parameters may be used to treat one or more conditions, such as dry eye. Ideally in the physicians' office the controller would run through a range of pre-established frequencies and patterns. This range is to determine an individual patient's best response of resonant frequency to the applied vibration. This resonant frequency is the condition best suited to drive an individual's flow of the meibomian lipids from the glands.

The tunable Resonant Frequency Vibrating Generator (RFVG) for the mask may be provided by a number of different sources including sonic generators, electrodynamic or mechanical (cell phone vibrators) vibration generators. It is important the source be relatively quiet and able to deliver the vibrational energy through the disposable patient contacting mask to the underlying tissue. And according to the concept of finding resonance to the patient's blocked oil glands the frequency must be adjustable and tunable. There are a number of miniature vibrating modules like the one from Adafruit, FIG. 6C, which have the required range for patient individual tuning capabilities. This particular module has a 0-5VDC range running at 11,000 rpm at the top end at 100 mA. The range can be reduced to 0, by reducing the voltage to the module, for example: 3VDC @ 60 mA is linear and at 60% or 6,600 rpm. The power requirement is very low and can be operated by remote supplied energy or in-eye-mask supplied battery power.

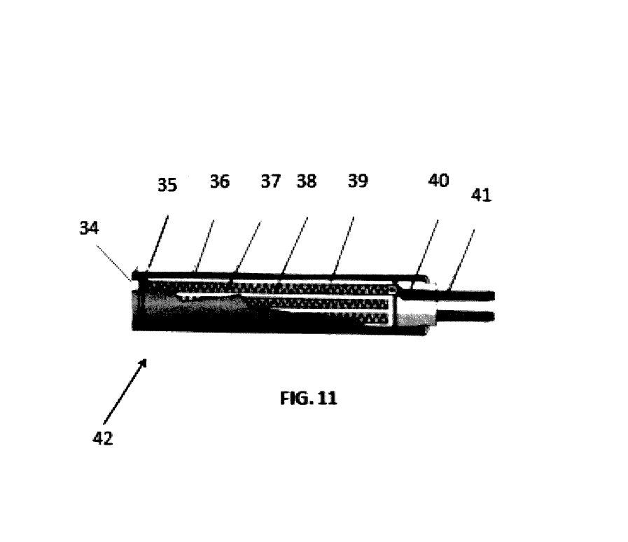

Direct heating of the eyelids and adjacent areas can be achieved by weaving a resistance Nichrome heater wire 13 into the reusable mask as shown in FIG. 6A. The energy to heat the reusable-mask to the desirable temperature will be provided by a USB connector 8. The resistance across the Nichrome heater wire dictates the heat converted by the amount of energy provided. The reusable mask 11 has a compartment, or slip on top of the heater area, for the imbedded resonant frequency generator 12. The heat control from the heater wire can be also be controlled by the same type device controlling the RFVG. Referring to FIG. 11, the patient's Meibomian gland can also be warmed effectively and comfortably by use of a far infrared front end spot heater 42. Far infrared radiation can be directed to a precise location and the target area warmed to a precise temperature of 40-43° C. Radiation is the most prevalent source of heat transfer in our universe and the Stefan-Boltzman law of radiation states that as the temperature of a heat source is increased, the radiant output increases to the fourth power of its temperature. This means that Far Infrared targeted heating is a logical approach to Meibomian gland care.

The far infrared front end spot heater 42 is constructed to radiate heat from the far infrared end seal 34 made of heat transmitting material (thin metal face or substitute). Heat is transferred to the far infrared end seal 34 by a conducting plug 35. This plug is in contact with the end seal 34 and is a designed mass of conducting material for storing and releasing the heat converted by a heating element wire 37. The exterior 36 or sides of the spot heater 42 are comprised of heat resistant insulation material allowing a user or to comfortably hold the spot heater without risk of uncomfortable temperature exposure. A thermocouple might also be employed with this device and integrated into the spot heater proper. The interior of the spot heater 42 includes a conducting packing material 38 all the way to the tip or plug 35 thru 40. The heating elements 37 are supported in the spot heater by ceramic element supports 39 that function in a stability capacity providing little movement and adding longevity to the spot heater device. The electrical leads are fed through a ceramic cap 40 providing support for the electrical leads and temperature barrier characteristics. The insulated electrical leads 41 are comprised of insulated electrical wire with lead lengths ending in a USB connector for operating the spot heater in the Doctors' office.

The reusable-mask 11 is comprised of soft, comfortable fabric like materials with an adjustable band to help the reusable mask reside in the appropriate location on the eyes. A moldable coupling device is required for the reusable mask to provide a sanitary, possibly sterile, skin contacting surface for individual patient use. This single use, disposable coupling device will transfer the generated thermal and vibration energy generated by the mask effectively to the eye lid surface.

The coupling device is composed of a hydrogel, similar to a hydrogel dressing, possibly contained in a support structure or quilted construction to assure even distribution and intimate contact across the skin contacting regions. The hydrogel composition and water are controlled to best achieve this transfer, and add a controlled amount of moisture to the eyelids and lashes, with the added benefit of loosening debris on the eye lashes. In a preferred embodiment the hydrogel layer makes direct skin contact. In alternate embodiments the hydrogel could be constrained behind a thin moisture permeable barrier layer.

Construction of the coupling device would allow hand molding to an individual's face and features or gentle reforming could be applied from pressure by the eye mask. The disposable coupling device would be easily replaceable in the reusable mask for use by a new patient. The coupling device would be prepared for long term storage using the barrier layer technologies described for the heating disk and could be sterilized to a 10⁻³ or higher sterility assurance level (SAL).

As Explained with the heating disk above, this hydrogel layer could incorporate a mixture of particles to facilitate well dispersed heat transfer, heat sinking and vibration energy transfer.

Alternatively the coupling device could be made from thin layers of natural materials and fibers to create a comfortable and breathable surface against the skin. The mask could be any number of fiber materials know to be breathable, cotton, linen, bamboo or hemp for example. Other cloth fabrics from synthetic materials are also breathable and moisture transportable. Examples include base layer clothing made from polyester and polypropylene. Filler materials inside the coupling device could be also made of breathable, natural fillers. The filler material must allow the heat to pass to the contact surface but also the vibration energy. Possible natural fillers, in small chunks or fibers, include bamboo fiber, small dried beans, quinoa, rice, hemp. Size and size distribution of the filler material can be optimized to determine best options for transmitting the vibration energy. Also possible are quilted fabric layers using various fillers to provide the loft in the quilt. Non-woven felt materials are possible.

Another feature of the coupling device would apply moist heat to the surface. A source for the moist water vapor could be the hydrogel. As heat energy from the mask transfers to the coupling device water in the hydrogel or natural filler turns to vapor and crosses a moisture permeable barrier to the contact surface.

Alternately, reservoirs of water could be constructed into the coupling device to interact with the heat source.

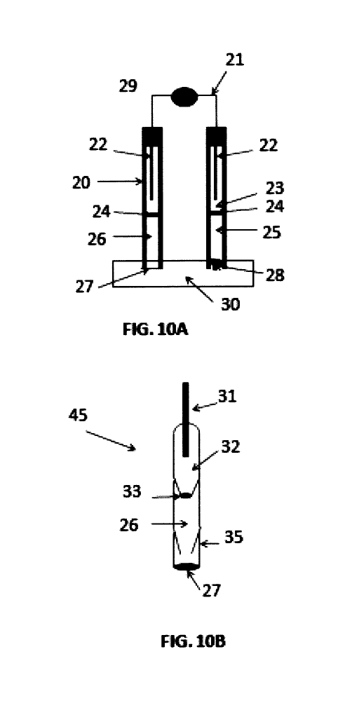

A micro fluidic enabled sensor can be included at the patient's eye lid interface and be responsive in real time to track changes in Meibomian fluid flow. Changes in flow rate are induced by variation in vibration frequency from the RFVG; the objective being to determine the best vibration parameters for an individual patient. The sensor provides analysis of very small samples and environments such as the Meibomian gland with the ability to measure very small change in flow. The chemical sensor, is an ultra-sensitive yet simple sensor integrated into a microfluidic device, incorporating polymer-based Meibomian fluid selective liquid-contact and polymer-based solution-selective electrodes. The target component in the Meibomian fluid for the sensor analysis could be specific proteins, lipids or other biomarkers produced with the flow of the fluid. In-situ sensors enable analysis of very small samples and environments such as the Meibomian gland at work with the ability to realize potentiometric output from very small changes of fluid flow utilizing liquid-contact electrodes FIG. 10A. It is possible to incorporate the miniature resonant frequency vibration generator and the miniature integrated chemical sensor into the same device.

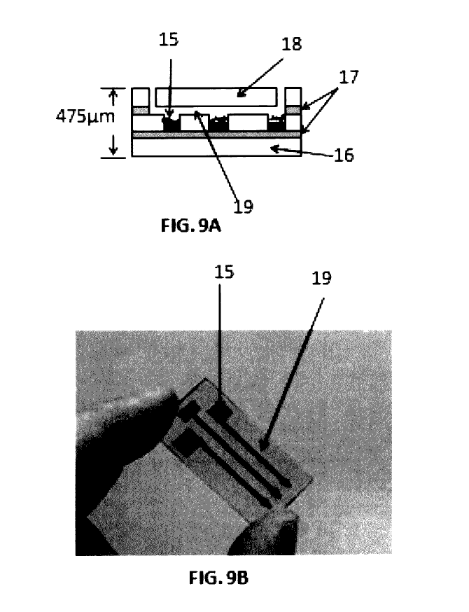

One form of the chemical sensor, FIG. 9, has a number of layers formed by Poly (methyl methacrylate) PMMA construction. This sensor has a lower PMMA layer 16 a top PMMA layer 18 a sensor liquid entrance, pressure sensitive adhesive (PSA) 17 conducting polymers, thermal pressure laminating and a CO₂ laser-400 μm width generated fluid flow channels 19. This version will require solution reactive material in the channels for detection 15. The deposition of solution reactive materials in the channeling is accomplished by electrode sputtering, if metallic. This process is well known in the art but has yet to focus on Meibomian gland issues as a target for patients until now. In this configuration the data recorded would be limited to the presence of fluid and no additional information.

Referring to FIG. 10A a miniature solution selective electrodes (SSE) with integrated dual chemical sensors 20 with potentiometric output is displayed. The chemical sensor uses a reference electrode 21 consisting of an inner reference half-cell 22, a reference solution 23, a diaphragm 24, bridge solution with a diaphragm, capillary or sleeve 28 at the entrance. The SSE consists of an inner reference half-cell 22, reference solution 23, diaphragm 24, inner filling solution 26 and a solution selective membrane 27. This miniature chemical sensor when placed on the surface of the Meibomian gland will detect for the duration of flow for patient diagnosis. Electric potential output from the SSE 20 will be connected to a data acquisition module able to retrieve data up to 100 Hz speeds, real-time. This will allow real-time data mapping of the patient's Meibomian gland fluid,

Proteins, biomarkers or lipids are tracked to define each patient's individual characteristics and medical needs.

A single element version of the miniature integrated chemical sensor 45 with potentiometric detection, FIG. 10B, is also applicable. It is comprised of a solution sensing wire material 31, a reference solution 32, a separation plug 33, inner filling solution 26, SSE body 45 and the selective membrane 27. This single element miniature integrated chemical sensor can be placed on the Meibomian gland and record data at the same rate as the dual sensor.

It is understood that most vibration and temperature are precisely controlled by Pulse Width Modulation (PWM. Meaning, nearly all switching-voltage regulators employ (PWM) control for the switching elements. The PWM signal is either generated from a control voltage (derived from subtracting the output voltage from a reference voltage) combined with a saw tooth waveform running at the clock frequency for the voltage-mode regulator, or by adding a second loop feeding back an inductor current for current-mode control. Modern devices have largely overcome the major drawbacks of older designs by employing techniques such as voltage feed-forward for voltage-control designs and slope compensation for current-mode units.

The result is a choice of both types of topology or the relationships and control between parts linked together in the system. Voltage-mode control switching regulators are recommended when wide-input line or output-load variations are possible, under light loads (when a current-mode control-ramp slope would be too shallow for stable PWM operation), in noisy applications (when noise from the power stage would find its way into the current-mode control feedback loop), and when multiple-output voltages are needed with good cross regulation.

Current-mode control devices are recommended for applications where the supply output is high current or very-high voltage; the fastest dynamic response is required at a particular frequency, input-voltage variations are constrained, and in applications where cost and number of components must be minimized as in the innovations stated here within.

As mentioned the mobile control for the reusable mask and entire system would be easily hand held and carried for patient use. This control may also be driven by smart phone (iOS, Android or Windows mobile as examples) or similar interfaces. Mobile medical interfaces are known for many uses and products like Zebra's MC40 Mobile Computer are available for this purpose. This type or other Wi-Fi, cell phone or Bluetooth connected interface can be used to control the patient's first in office use of the system where a range of frequencies are tested and store output data from the sensors. This data storage and associated algorithm can determine the best treatment mode for following office visits or transfer an optimal program to an at-home unit. These mobile interfaces further create efficiencies for the office staff by automatically storing patient records to the electronic medical records (EMR) of the first and subsequent uses. These records include patient name, time and date of use, frequencies explored and sensors output during that time. The at-home unit would also serve as a record of patient compliance to prescribed therapy.

The harmonic resonance heating mask is preferentially supplied as a kit. Kits include one or more devices, and varying numbers of replacement heaters depending on kit size. Kits can include both elements of the one-time use components and reusable components. For example: a kit might include the one-time use heater element, the reusable miniature harmonic resonance frequency generator pairs that fit into the eye patch component and plug into a USB port and the one-time use coupling device. Kits can be provided to a patient during an office visit as the equipment used to define the correct resonance frequency would be available in the practitioner's office.

Commercial kits could also be provided with very specific frequencies and then purchased directly by an informed customer.

As mentioned in the discussion of the mobile controller, after a patient's first use of the system in the Physician's office, the patient may be prescribed to continue therapy on a more frequent basis at home. As an alternate embodiment this system could be simplified for the home user. This system would have a reusable mask with built in heaters and resonance frequency generators, accommodate a disposable coupling device and come with appropriate power supply and control, including a mobile and wirelessly connected controller. The home use system would not require a full range of vibration frequencies as the optimal frequency and pattern was determined in the original office use and that pattern is programmed into the individual user's system. Similarly the sensing capability is not needed for home use. The cell phone, Wi-Fi or Bluetooth connected controller will also create a record of use for the patient's EMR. Patterns of noncompliance or misuse could create an alert to go directly to the patient or back to the treating physician.

A further alternate embodiment may include a system that employs single use heating discs described earlier. This could be used for either the office based or home use products. The disposable heating disc, being hand moldable to conform to an individual's anatomy, would fit into the pocket in the mask. This heating disc element could also be built into and supplied as part of the coupling device that contacts the skin and comprises a combined single disposable item. As shown in FIG. 3A & 3B, a moldable heating disc with a low cost RFVG may located in disposable heating disc and connected via the USB port for control and energy. A reusable RFVG (mini-vibrator) can be placed in the manufactured pocket location located in the moldable heating disc. This approach may be used for both the moldable gel and polymer eye piece.

The use of the device and different embodiments described comprises a method of treating dry eye disease or meibomian gland dysfunction. These methods include the initial physician's office based use where optimal treatment parameters are determined and then stored for later use either in subsequent office visits or home use.

The advantages of the devices disclosed include, without limitation that it is portable, easy to transport, reliably functions as intended, and simple and convenient to activate and use. It is easy to integrate these devices into a reusable face mask or eye patch because they are relatively small and lightweight, showing the parallel utility of the device components stated herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that any claims presented define the scope of the invention and that methods and structures within the scope of these claims and their equivalents be covered thereby.

A further alternate embodiment may include an integrated real-time imaging device to detect optimal tuning of the RFVG to the particular patient eyelid and Meibomian glands. For example, Optoacoustic imaging, or photoacoustic imaging, is insensitive to photon scattering within biological tissue and, unlike conventional optical imaging methods, makes high-resolution optical visualization deep within tissue possible. A key empowering feature is the development of video-rate multispectral imaging in two and three dimensions, which offers fast, spectral differentiation of distinct photo-absorbing moieties (Nature Photonics, 9, 219-227 (2015)). The imaging device provides a real-time image based assessment of the optimal settings for the miniature resonant frequency vibration generator at which there is maximal movement of the eyelids, Meibomian glands, and lipid fluid within the Meibomian glands.

References:

1. “Understanding and Applying Current-Mode Control Theory—Practical Design Guide for Fixed-Frequency, Continuous Conduction-Mode Operation,” Robert Sheehan, National Semiconductor, October 2007.

2. “Voltage-Mode, Current-Mode (and Hysteretic Control),” Sanjaya Maniktala, Microsemi, TN-203, 2012.

3. “Switching Power Supply Topology Voltage Mode vs. Current Mode,” Robert Mammano, Unitrode, DN-62, June 1994.

4. “Modelling, Analysis and Compensation of the Current-Mode Converter,” Texas Instruments, U-97, 1999.

5. Stimulation devices and Methods, Ackermann et al, Date Oct. 11, 2012

Part List

1. Heating disk

4. Barrier layer

5. Pouch Tear strip

6. Resonant Frequency Vibration Generator

7. Lead wire to Resonant Frequency Vibration Generator

8. USB connector

9. Aluminum or metal nano-particles infused into the Heater Disc

10. Ceramic or silica nano-particle material

11. Reusable Mask

12. Imbedded Resonant Frequency Vibration Generator (RFVG)

13. Electrical heating elements

14. Resonant Frequency Vibration Generator with pulse modulation controller

15. Meibomian gland fluid selective catalyst layer

16. Lower PMMA layer

17. PSA sealing layers

18. Top PMMA Layer

19. Sensor Fluid/Gas flow path

20. SSE Body (Solution Selective Electrodes)

21. Reference electrode

22. Inner reference half cell

23. Reference solution

24. Diaphragm

25. Bridge solution

26. Inner filling solution

27. Solution-selective membrane

28. Diaphragm, capillary or sleeve

29. SSE (Solution Sensing Electrode)

30. Sample, fluids, tears

31. Solution sensing wire or substitute

32. Reference solution

33. Plug

34. Far Infrared end seal (metal)

35. Conducting plug (Mass for heat sink)

36. Insulation, heat resistant

37. Element wire

38. Conducting packing to tip

39. Ceramic element support

40. Ceramic cap

41. Insulated electrical leads

What is claimed is:

1. A moldable heating disc with miniature harmonic resonant frequency vibrating generator comprising:
A single or multiple moldable heating disc or elements and
A Single or multiple miniature resonant frequency vibrating generator or generators and
A moldable coupling device or devices and
A reusable mask made to hold the moldable heater element or elements, miniature harmonic resonance frequency generator or generators and moldable coupling device or devices for use in parallel utility of the devices.
An optional ultra-sensitive sensor array to determine tuning parameters of the vibration and heating profile for the individual patient eyelid and periorbital 3 dimensional anatomy.

2. The moldable heater article in independent claim 1, wherein said moldable heater elements are comprised of a polymer with pliable but strong moldable material and high shape retention characteristics.

3. The moldable heater article in 2 wherein said moldable heater is a single use heating disc wherein the element polymers are manufactured providing the heater elements with a 20-80% pore volume resulting in high porosity in the moldable heater structure.

4. The moldable heater article in 3 wherein the said high porosity polymer heater element or elements are infused with FeOₓ heating material.

5. The moldable heater article in 4 wherein the said FeOₓ heating material having the reaction (4Fe(s)+3O₂(g) >>2Fe₂O₃(s) and is comprised of specific ratios of the ingredients and manufactured to produce the precise heater temperature for the specific time duration of 10-20 minutes necessary for optimal patient therapeutic effect.

6. The moldable heater article in independent claim 1 wherein the said moldable heater element is manufactured containing a defined pocket indentation for locating the miniature resonant frequency vibrating generator.

7. The miniature resonant frequency vibrating generator article in Independent claim 1, 6 wherein the said miniature resonant frequency vibrating generator is preset to a specific harmonic resonance frequency for optimal patient therapeutic effect.

8. The miniature resonant frequency vibrating generator article in 7 wherein the said preset harmonic resonance frequency generator is comprised of frequencies of 2 Hz-270 Hz, 15 Hz-40 Hz or 30 Hz-60 Hz.

9. The miniature resonant frequency vibrating generator article in 8 wherein the said preset harmonic resonance frequency for each patient is determined by the Health Care provider.

10. The miniature resonant frequency vibrating generator article in 9 wherein the said preset harmonic resonance frequency is determined using an in office, variable harmonic resonance frequency generator able to test the available resonance frequencies to identify the individual patient's optimal therapeutic frequency for tear stabilization and flow of meibom from the Meibomian glands.

11. The miniature resonant frequency vibrating generator article in 7 wherein the said device incorporates a miniature, ultrasensitive biochemical sensor capable of detecting the optimal tissue response to the harmonic resonance frequency.

12. The miniature resonant frequency vibrating generator article in 7 wherein the said device incorporates a miniature, ultrasensitive biochemical sensor used to map the biochemical makeup of the Meibom lipids.

13. The miniature resonant frequency vibrating generator article in 7 wherein said miniature resonant frequency vibrating generator incorporates a length of 0.5 cm to 2.5 cm. and thickness of the device is 1 mm to 5 mm with a usable width of 2 mm to 10 mm.

14. The moldable heater with miniature resonant frequency vibrating generator article in Independent claim 1, 7 wherein the said device comprising heater elements and harmonic resonance frequency generators are secured to the face using an eye patch or reusable mask when utilizing both heaters and miniature resonant frequency vibrating generator.

15. The eye patch or eye mask article in 14 wherein the said eye wear is comprised of breathable cotton, linen, bamboo or hemp.

16. The eye patch or reusable mask article in 15 wherein the said eye wear contains the miniature resonant frequency vibrating generator.

17. The eye patch or reusable mask article in 16 wherein the said eye wear comprises one or two pockets for locating the heating disc element without the miniature resonant frequency vibrating generator.

18. The eye patch or reusable mask article in 17 wherein the said eye wear is comprised of material, 15 enhanced with vibration transfer facilitating material such as metal wire woven into the fabric. Drawing 3A.

19. The eye patch or reusable mask article in 18 wherein the said eye wear contains an electrical lead with a connector such as a USB connector to drive both the miniature resonant frequency vibrating generator and the permanent heater in the fabric as shown in drawing 3A.

20. The moldable heating disc article in independent claim 1, 5 wherein the said moldable heating disc comprises nano-particles of ferrous or non-ferrous metals to facilitate both heat transfer and harmonic resonance frequency transfer to the Meibomian glands as seen in FIG. 4A and FIG. 4B.

21. The moldable heating disc article in claim 20 wherein the said moldable heater comprises nano-particles of ferrous or non-ferrous metals and nano-ceramic particles to facilitate heat transfer, resonant frequency vibration transfer and control heat transfer rate to the Meibomian glands as seen in FIG. 5A and FIG. 5B.

22. The moldable coupling device article in independent claim 1, wherein the said moldable coupling comprises a coupling agent material such as Hydrogel used to transfer the harmonic vibration energy or movement to the surface of the eyelid and/or the Meibomian gland.

23. The moldable coupling device article in 22 whereas the said moldable coupling agent transmits the mechanical response to the eyelid and/or the Meibomian Gland.

24. The moldable coupling device article in 23 whereas the said moldable coupling agent is a single use, disposable sterile or non-sterile component.

25. The moldable coupling device article in 22 wherein the said moldable coupling comprises a coupling agent with a solution sensor integrated into the coupling device, and recording the response.

26. moldable coupling device article in claim 25 wherein the said moldable coupling comprises a coupling agent with an integrated piezoelectric device used to record the response and compare said response to patient subjective impression of most effective/pleasing frequency.
